(19) United States
(12) Patent Application Publication
(10) Pub. No.: US 2002/0099363 A1
(43) Pub. Date: Jul. 25, 2002

(54) Radiation treatment system and method of using same

(76) Inventors: Benjamin W. Woodward, Santa Clara, CA (US); Satish V. Herekar, Palo Alto, CA (US); Mark S. Milner, Woodbridge, CT (US)

Correspondence Address: K. Alison de Runtz, c/o SKJERVEN MORRILL MacPHERSON LLP, 28th Floor, Three Embarcadero Center, San Francisco, CA, 94111, US

(21) Appl. No.: 09/770,094

(22) Filed: Jan. 23, 2001

(51) Int. Cl. A61F 7/12; A61B 18/18

(52) U.S. Cl. 606/5; 607/96; 607/101

(57) ABSTRACT

The present invention provides a system and associated method for advantageously treating bodily tissue containing collagen, such as ophthalmic tissue. The treatment includes providing a pattern of heating to which the tissue is to be exposed and the exposure of that tissue to shrink collagen therein. In ophthalmic applications, the pattern includes at least one region that corresponds to an ocular region of from about 4 mm to about 20 mm radially from the optical axis. The shrinkage of collagen within this ocular region improves accommodation for near vision. The present invention thus provides an effective treatment for the ocular condition of presbyopia.

### CROSS-REFERENCE TO RELATED APPLICATIONS

This application is related to co-pending U.S. patent application Ser. No. 09/146,999 (hereinafter, “First Co-Pending Application”) of Herekar et al., filed on Sep. 4, 1998, and entitled “METHOD AND APPARATUS FOR EXPOSING A HUMAN EYE TO A CONTROLLED PATTERN OF RADIATION”, as well as to a co-pending, continuation-in-part application related thereto, U.S. patent application Ser. No. 09/388,635 (hereinafter, “Second Co-Pending Application”) of Herekar et al., filed on Sep. 1, 1999, and entitled “RADIATION TREATMENT SYSTEM AND METHODS OF USE FOR TREATING EYES TO CORRECT VISION”. This application is also related to a co-pending U.S. patent application Ser. No. 09/376,269 (hereinafter, “Third Co-Pending Application”) of Herekar, filed on Aug. 18, 1999, and entitled “APPARATUS AND METHOD FOR PREPARING AN EYE OF A SUBJECT, AND SYSTEM AND METHOD FOR TREATING EYE TISSUE OF A SUBJECT”; a co-pending U.S. patent application Ser. No. 09/559,933 (hereinafter, “Fourth Co-Pending Application”) of Haghighi, filed on Apr. 27, 2000, and entitled “COMPOSITIONS AND METHODS FOR STABILIZING MODIFIED TISSUE”; and a co-pending U.S. patent application Ser. No. 09/588,274 (hereinafter, “Fifth Co-Pending Application”) of Woodward et al., filed on Apr. Jun. 6, 2000, and entitled “OPTICAL DEVICE AND FLUOROMETRY SYSTEM AND METHODS OF USING SAME”. This application is further related to a co-pending U.S. patent application Ser. No. ___/___,___ (hereinafter, “Sixth Co-Pending Application”) of Haghighi, filed concurrently herewith, and entitled “Apparatus and Method for Shrinking Collagen”. These First, Second, Third, Fourth, Fifth and Sixth Co-Pending Applications, including any microfiche appendix thereof, are expressly incorporated herein in their entireties by this reference.

### REFERENCE TO A MICROFICHE APPENDIX

A microfiche appendix including four microfiche containing a total of 186 frames forms a part of the disclosure herein.

### FIELD OF THE INVENTION

The invention relates generally to a system for treating the eye of a subject, and a method of using same. More particularly, this invention relates to a preferably coordinated or automated system that is used to prepare the eye of a patient for medical treatment and to treat the eye, and methods associated with such a system. The system and method of using same are particularly useful in vision-modification applications, such as photothermal keratoplasty applications wherein a defined pattern of electromagnetic radiation is delivered to an eye to address a condition of presbyopia. Although the various aspects of the present invention are primarily described with respect to ocular tissue, they also have application to the treatment of bodily tissue other than found in the eye, such as to reshape an outside surface of such tissue.

### BACKGROUND OF THE INVENTION

There are many specific treatment procedures which involve directing a highly controlled beam of electromagnetic radiation to an eye. For example, one specific surgical procedure involves using a radiation beam to ablate and thus cut portions of the corneal tissue. A specific application of this surgical procedure is in the performance of a radial keratotomy procedure, in which radial cuts are made in the cornea using a laser as opposed to a surgical knife. In another specific treatment procedure, an outside surface of the cornea is removed by an excimer laser in order to reshape the cornea. Despite the existence of the aforementioned specific procedures, alternative “keratoplasty” procedures are currently receiving a great deal of attention because of their ability to correct for myopia (near-sightedness), hyperopia (far-sightedness), and/or astigmatism.

In a particular keratoplasty procedure, which avoids cutting the cornea, at least one beam of electromagnetic radiation within the infrared portion of the spectrum is directed at the eye to shrink collagen tissue within the cornea in order to cause corrective changes in corneal curvature. This technique, often termed “photothermal keratoplasty”, is the subject of the aforementioned Sand Patents and of U.S. Pat. No. 5,779,696 to Berry et al. (hereinafter, the “Berry et al. Patent”). The aforementioned Sand Patents and the Berry et al. Patent are expressly incorporated herein in their entireties by this reference.

The collagen-shrinkage methods and apparatus of Sand and Berry et al. are disclosed as being applicable for modification of collagen tissue throughout the body. When the tissue is corneal collagen tissue and the radiation source is a laser, such methods are typically referred to as “laser thermokeratoplasty” or “laser thermal keratoplasty” (LTK). These LTK techniques promise to provide permanent changes to the optical characteristics of the human cornea with a higher degree of safety and patient comfort than that provided by techniques that involve physically cutting and removing portions of the cornea.

One way to deliver a desired electromagnetic radiation pattern to the cornea is by projection from a short distance removed from the cornea. One instrument for doing so is described in the Published International Patent Cooperation Treaty Application WO 94/03134 (hereinafter, the “PCT Publication”), which PCT Publication is expressly incorporated herein in its entirety by this reference. This instrument allows an ophthalmologist, or other attending physician or practitioner, to select and deliver a specific pattern and amount of electromagnetic radiation to each patient in accordance with the condition to be corrected. It is desirable for such an instrument to perform efficient corrective photothermal keratoplasty procedures on a large number of patients with a high degree of accuracy, effectiveness, safety and convenience.

It is also desirable to make the exposure system as automated as reasonably possible for determining a pattern of radiation exposure that is appropriate to correct the vision of a particular eye of a patient, for preparing the eye for such an exposure and for then exposing the patient's eye to that pattern of radiation, all with convenience and efficiency for an attending physician or other provider of the treatment. The aforementioned First Co-Pending Application discloses apparatus and methods for advantageously exposing an eye of a patient to a controlled pattern of radiation, while the Second Co-Pending Application discloses coordinated or automated apparatus and methods for so exposing the eye, to provide convenience and to promote efficiency for an attending physician or other provider of the treatment.

The aforementioned Third Co-Pending Application discloses apparatus and methods for applying a flow of a conditioning or drying medium to an external surface of an eye of a patient, to dry the eye in preparation for ophthalmological observation and/or treatment. Such apparatus and methods are particularly useful in preparing a patient's eye for vision-corrective ophthalmological treatments, such as photothermal keratoplasty or LTK. The aforementioned Fourth Co-Pending Application discloses compositions and methods useful to stabilize a condition of collagenous tissue that results from its modification. The aforementioned Fifth Co-Pending Application discloses optical devices, systems, and methods useful to determine a condition of collagenous tissue, and particularly useful for developing a process for modifying such tissue.

It is desirable to have a versatile exposure system that may be used to treat a variety of ophthalmic conditions, such as the aforementioned myopia, hyperopia, and astigmatism, as well as other ophthalmic conditions, such as presbyopia. There are no known exposure systems that are specifically directed to the treatment of presbyopia, or are easily modified for the treatment of presbyopia.

Presbyopia refers to a condition of compromised ability or inability to accommodate for near vision, when distance vision is naturally emmetropic or emmetropic by virtue of some form of correction, such as corrective lenses or refractive surgery. Presbyopia may also be described as the loss of amplitude of accommodation for near vision. See, for example, Schachar, Ann. Ophthalmol. 24, pp.445-52 (December 1992). Accommodation for near vision is normally accomplished by changes in the shape or possibly the position of the crystalline lens, such as a central thickening of the lens to create a greater surface curvature or possibly a movement of the lens in an anterior direction, which changes tend to bring the focal point nearer the retina when objects are nearer the viewer than infinity. There are various theories as to the manner in which the lens shape, and possibly position, changes in response to a near object, such as the Helmholtz theory, and more recently, the Schachar theory. See Gilmartin, Ophthalmic Physiol. Opt. 15, pp.431-7 (September 1995); Atchison, Ophthalmic Physiol. Opt. 15, pp.255-72 (July 1995); Schachar, J. Fla. Med. Assoc. 81, pp.268-71 (April 1994); and Wilson, Trans Am. Ophthalmol. Soc. 91, pp.401-419 (1993).

According to the Schachar theory, presbyopia is caused by a normal increase of the equatorial diameter of the lens and a consequent decrease in the distance between the lens and the ciliary muscle of the eye. See, for example, Schachar, Ann. Ophthalmol. 24, pp.445-52 (December 1992). Following this theory, Schachar has sutured scleral enlargement band segments (SEBS) to the sclera to enlarge the space available for ciliary muscle action. See Schachar, U.S. Pat. No. 5,465,737. Reportedly, the SEBS have had at least short-term success, giving some validation to the Schachar theory. Unfortunately, as the SEBS are sutured to the sclera of the patient, they require significant time, skill, and labor of the surgeon and are invasive to the patient.

There is a need for a substantially non-invasive system that may be used to treat a variety of ophthalmic conditions, such as the aforementioned myopia, hyperopia, and astigmatism, as well as other ophthalmic conditions, such as presbyopia.

### SUMMARY OF THE INVENTION

The present invention is directed to a system and an associated method for treating ocular tissue of an eye to improve the accommodation for near vision. The treatment includes providing a pattern of heating according to which the ocular tissue is to be exposed, and exposing the tissue to such heating sufficient to shrink collagen within the ocular tissue. The pattern of heating includes at least one region of heating that corresponds to an ocular region defined by a radial distance of from about 4 mm to about 11 mm from the optical axis. This region includes a corneal region and a scleral region, from about 4 mm to about 6 mm and from about 6 mm to about 11 mm, respectively. It is believed that treatment according to the present invention in any one or both of these regions causes a tensile force that is directed outwardly from the eye, which in turn induces motion of corneal, scleral and/or limbal tissue in an outward direction, away from the crystalline lens of the eye. When the corneal, scleral and/or limbal tissue is so moved, an improvement in near vision accommodation is observed.

The apparatus for carrying out the method of the present invention is preferably quite similar to that described in the First and Second Co-Pending Applications mentioned above. Such a system is preferably used to provide infrared laser energy in a pattern of spots, such as eight spots, simultaneously, to the targeted tissue. Preferably, the system is modified to facilitate treatment at diameters greater than those associated with treatments of hyperopia, namely, the diameters associated with treatments of corneal, scleral and/or limbal tissue set forth above. Such a modification may include the use of a polyprism having angled surfaces that are more steeply angled than those associated with the polyprism used for hyperopic treatments, or a polyprism that may be adjusted to provide angled surfaces of a desired steepness for a particular treatment.

The present invention provides an effective apparatus and method for treating a condition of presbyopia by improving near vision accommodation. While the invention is most often described in relation to this application, it can be used in the preparation and/or treatment of a variety of substrates, such as non-corneal or non-ophthalmic tissue. Additional aspects, advantages and features of the present invention will become apparent from the description of preferred embodiments, set forth below, which should be taken in conjunction with the accompanying drawings, a brief description of which follows.

### BRIEF DESCRIPTION OF THE DRAWINGS

The drawings include FIGS. 1-13, not all of which are drawn to scale or to the same scale. These drawings are briefly described below.

FIG. 1 is a perspective view of an ophthalmic treatment system for producing controlled patterns of treatment radiation, as disclosed in the aforementioned First Co-Pending Application, wherein the view is from a side that faces an attending physician.

FIG. 2 is a close-up, perspective view of a portion of the ophthalmic treatment system of FIG. 1, wherein the view is from a side that faces a patient and a patient headrest is removed for better viewing.

FIG. 3 is a simplified schematic diagram of the optical system within the ophthalmic treatment system of FIG. 1.

FIG. 4 is a simplified drawing of a typical human eye.

FIG. 5 is a schematic illustration of an exposure produced by the ophthalmic treatment system of FIG. 1, which exposure may be used in connection with a human eye, such as that shown in FIG. 4.

FIG. 6 is a schematic representation of the optical arrangement of the ophthalmic treatment system of FIG. 1.

FIG. 7 is an example screen display used to develop a treatment radiation pattern for treatment of a specific patient's eye.

FIG. 8 is an illustration of a treatment plan pattern of radiation spots arranged in a circular pattern.

FIG. 9 is a table that shows information display fields that may be displayed to the user using the system computer and software therefor.

FIG. 10 is a diagram of a human eye, shown in vertical cross-section along a central axis of the eye, as viewed from the side.

FIG. 11 schematically illustrates treatment of an anterior portion of the human eye shown in FIG. 10, according to the present invention. The vectors shown in FIG. 11 are merely illustrative, and thus, are not intended to represent precisely their magnitude, direction, and geometry, which may vary according to parameters of a selected treatment.

FIG. 12 is a schematic illustration of exposures produced by the ophthalmic treatment system similar to that shown in FIG. 1, which exposures may be used in connection with a human eye, such as that shown in FIG. 4.

FIG. 13 is a schematic illustration of a polyprism element which may be used in the optical arrangement of FIG. 6 and a treatment pattern resulting therefrom, according to an embodiment of the present invention.

### DESCRIPTION OF SPECIFIC EMBODIMENTS

The invention is now described with reference to the above-described Figures. Reference symbols are used in the Figures to indicate certain aspects or features shown therein, with reference symbols common to more than one Figure indicating like aspects or features shown therein. It should be noted that reference symbols used herein are intended to be internally consistent, such that whether or not they happen to coincide with those used in applications that have been incorporated herein by reference, their meaning will be apparent to those of ordinary skill in the art.

The various aspects of the present invention, those summarized above and others, are illustrated herein to be implemented in a system that corrects vision by photothermal keratoplasty. The system is easily configured to precisely generate a desired pattern of electromagnetic radiation to correct a vision deficiency, such as farsightedness, of a particular patient. The specific type or amount of vision correction required by the particular patient determines the specific configuration for that patient. Many aspects of the present invention are also applicable to other techniques of eye vision correction, wherein certain parameters are different, such as the treatment radiation wavelengths, patterns, exposure times, and the like. Further, many aspects of the present invention are applicable to the generation of radiation patterns for other uses than correcting vision. Additionally, many aspects of the present invention are applicable to operation of a wide variety of medical treatment or diagnosis systems.

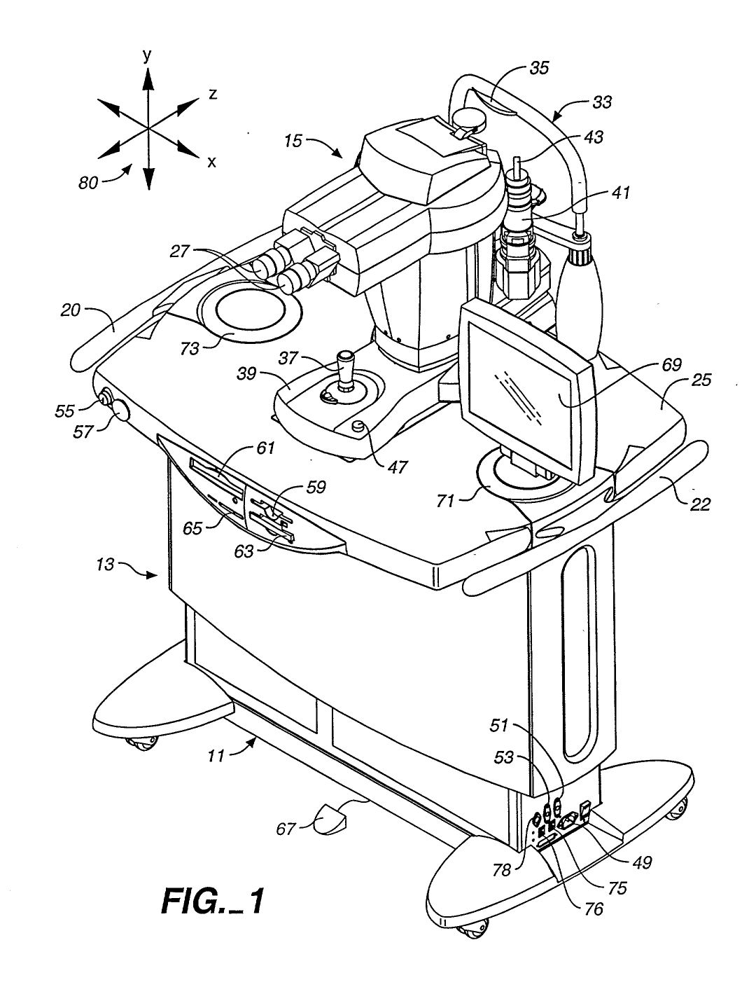

The example instrument will initially be generally described with respect to FIG. 1. By way of convenience, the system is described herein with reference to terms which correspond to a representation 80 of a three-dimensional Cartesian coordinate system, including an x-axis, a y-axis, and a z-axis. Right, left, lateral, horizontal, or like movement is in a direction substantially parallel to the x-axis; up, down, elevational, vertical, or like movement is in a direction substantially parallel to the y-axis; and fore, aft, proximity-adjusting, or like movement is in a direction substantially parallel to the z-axis.

This instrument is specifically designed for use in an office of an ophthalmologist, other physician or medical service provider, where reliability and ease of use are important since technical assistance is not on site or very close to the office. A base 11 is provided with casters for ease of movement of the system within the office. A table assembly 13 is carried by the base in a manner to be adjustable up and down with respect to the base by a motor (not shown) within the base. This allows vertical adjustment of an optical radiation delivery instrument 15 to suit a physician 17 that is performing the procedure and a particular patient 19 who is having his or her vision corrected. This adjustment, along with a usually independent adjustability of physician and patient chairs 21 and 23, permits comfortable positioning of both the physician and patient with respect to the instrument 15. Handles 20 and 22 on opposite sides of a top 25 of the table of the assembly 13 make it easy to move the system by rolling on its casters.

The radiation delivery instrument 15 is carried on the top 25. During the procedure, the physician looks through binoculars 27 on one side of the instrument 15 and a treatment optical radiation pattern exits the other side of the instrument through an opening 29. This radiation is directed through a few inches of air to a patient eye 31 being treated. Only one eye is treated at a time in one procedure. In order to hold the treated eye in a fixed position with respect to the table assembly 13, a headrest assembly 33 is attached to the table top 25. The headrest assembly 33 is described in more detail in a Published International Patent Cooperation Treaty Application WO 00/13751 (hereinafter, the “Herekar et al. PCT Publication”), which Herekar et al. PCT Publication is expressly incorporated herein in its entirety by this reference.

Briefly, in one operational embodiment, the patient's head is placed in contact with the assembly 33 in preparation for or during treatment. The head is optionally urged against the assembly 33 such as by being strapped against it. A transducer 35 is built into a top of the headrest assembly 33 in a position to be contacted by the forehead of the patient. This transducer provides an electrical signal with a magnitude related to a degree of contact between the patient's forehead and the assembly 33. By way of example, the degree of contact, and thus, the electrical signal, may be related to an amount of pressure or force applied to the assembly 33 when the patient's forehead contacts the assembly. The resulting electrical signal is used to confirm an appropriate level of contact, or to indicate an inappropriate level of contact, between the patient's forehead and the headrest assembly. Thus, this electrical signal is usefully fed into an electronic control portion of the system that, for example, may provide a desired safety response.

Once the patient's head is placed against the headrest assembly 33, the radiation pattern from the opening 29 is manually aligned with the eye 31 by movement of the optical instrument with respect to the table top 25. The physician so moves the instrument by manipulating a joystick type of handle control 37 on a base 39 of the instrument. The handle 37 operates a mechanism (not shown) positioned under the base 39 of the instrument 15 that, in response to movement of the handle 37 to the left or right by the physician 17, moves the projected radiation pattern between the patient's right and left eyes and horizontally adjusts the pattern on the selected eye 31 being treated. Movement of the handle 37 forward and backward by the physician 17 moves the instrument 15 toward and away from the patient, respectively, to control the focus of the radiation pattern on the eye 31 being treated. Vertical motion of the instrument 15 with respect to the table top 25 is not provided in this example, but could also be provided. Rather than moving the instrument 15 up and down with respect to the table top 25, the vertical position of the patient eye 31 being treated is controlled by a mechanical adjustment of the headrest assembly 33, as described in the above-referenced co-pending application.

Included as part of the optical instrument 15 is an illuminator 41 that directs light through a top prism 43 to the patient eye being treated from a side of the eye. This illuminates the eye so that the physician 17 may have a clear view of it through the binoculars 27 when carrying out the treatment procedure. The illuminator 41 is rotatable by hand with respect to the instrument 15 about an axis (not shown). The attending physician may easily adjust the angle of the eye illumination, while looking through the binoculars, in order to obtain a good view of the eye being treated. The illuminator 41 will generally be rotated to one side or the other, depending upon whether the right or left eye of the patient is being treated. Since the prism 43 directs light from about the same height as the treatment radiation output 29, it is rotated out of the way when treatment radiation is directed against the patient eye 31. The intensity of light from the illuminator 41 is adjusted by the physician through rotation of a knob 47 on the base 39 of the instrument. Alternatively, an illuminator may be housed within an optical instrument (not shown) which is equipped with appropriate illuminator controls, such as a modified optical instrument 15.

The base 11 includes a number of electrical receptacles for connection to power and communications systems. Included are a receptacle 49 for a power cord, a receptacle 51 for a telephone line and a receptacle 53 for a local area network (LAN). Several controls and devices are provided on the physician's side of the table assembly 13. These include a key-operated power switch 55 and an emergency button 57 that turns off the treatment radiation source. A floppy-disk drive 59 is also positioned on a side of the table facing the physician. A compact-disk (CD) drive 61, a high-capacity, removable-disk drive 63 and a slot of a card interface 65 for removably receiving an electronic card are also provided. Many of the radiation sources used in the system and a controlling computer are installed in the base unit 11. A foot switch 67 is provided for the physician to use to start treatment after the system is adjusted for a particular patient eye.

A primary input/output device to the system's controlling computer system is a touch-sensitive screen 69. It can be mounted to the table top 25 on either the right (as shown) or left side of the physician, by attachment to respective receptacles 71 and 73. Thus, the attending physician may select whichever side is the most convenient. A usual computer keyboard may also be connected to the internal computer system through a receptacle 75 in the base unit 13 but will unlikely be used by the physician to perform treatments since the touch screen 69 is usually preferred. A tray (not shown) can be added to extend the table top 25 to support a keyboard. A keyboard will be useful when a significant amount of data are input or retrieved through the treatment system, rather than though another computer connected in a LAN with the treatment system. Standard computer-peripheral receptacles 76 and 78 are also provided for connection to an external printer and monitor, respectively.

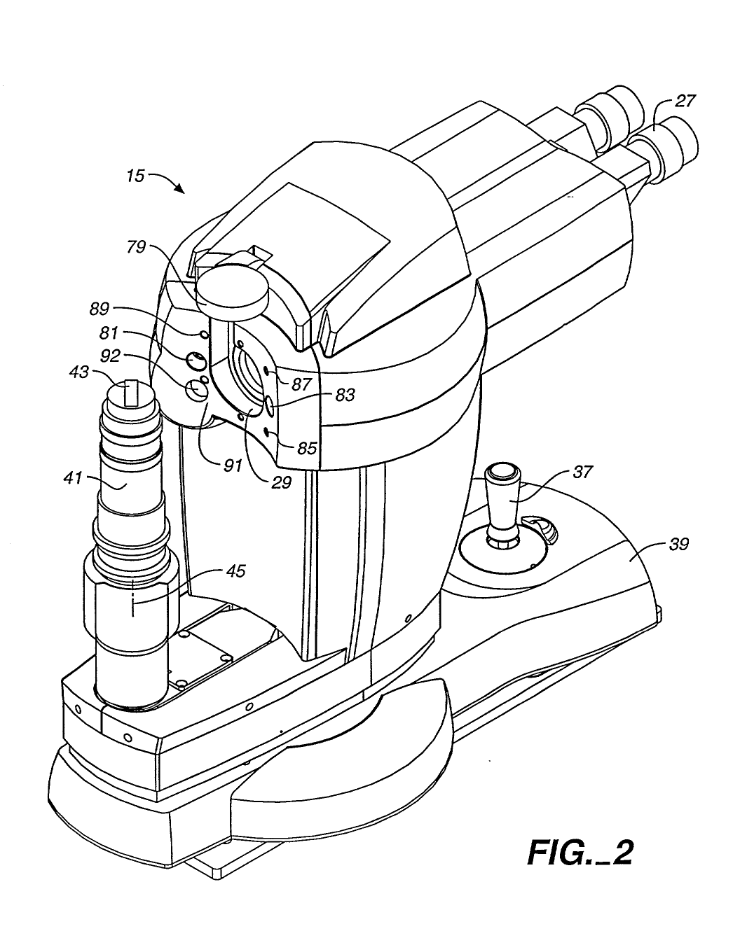

Referring to the enlarged view of the patient's side of the optical instrument 15 in FIG. 2, with the headrest removed, a radiation detector assembly 79 is provided for calibration prior to treatment. The assembly 79 rotates from an open position shown in FIG. 2, which allows the treatment radiation to be directed against the patient eye through the opening 29, to a closed position where the assembly 79 covers the opening 29. When in the closed position, the entire treatment radiation pattern is directed from within the instrument 15 onto a single radiation detector mounted on the inside of the assembly 79. Two spot projectors 81 and 83 are positioned on either side of the opening 29 to aid in focusing the instrument. Each spot projector focuses an output from an optical fiber onto the cornea. An input to these optical fibers can be any bright light source (not shown), such as a green helium neon (HeNe) laser or a green diode pumped frequency doubled neodymium doped yttrium aluminum garnet (Nd:YAG) crystal laser. Four light emitting diodes (LEDs) 85, 87, 89 and 91 are also positioned at comers of a square symmetrically positioned around an outside of the opening 29. These LEDs are usable to calibrate an eye tracker feature that may be included as part of the system. When included, an eye tracker camera 92 views the eye being treated and provides an electronic signal related to the position of the eye. It is particularly useful to monitor any motion of the eye during treatment. Eye tracker systems are commercially available.

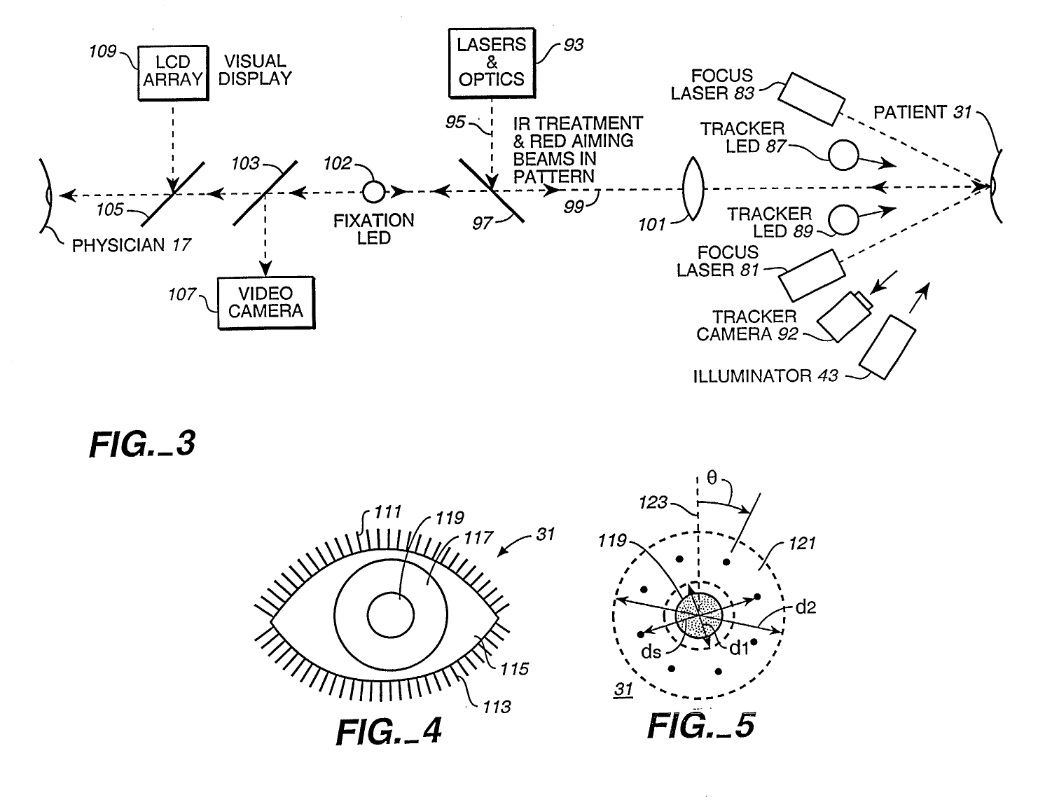

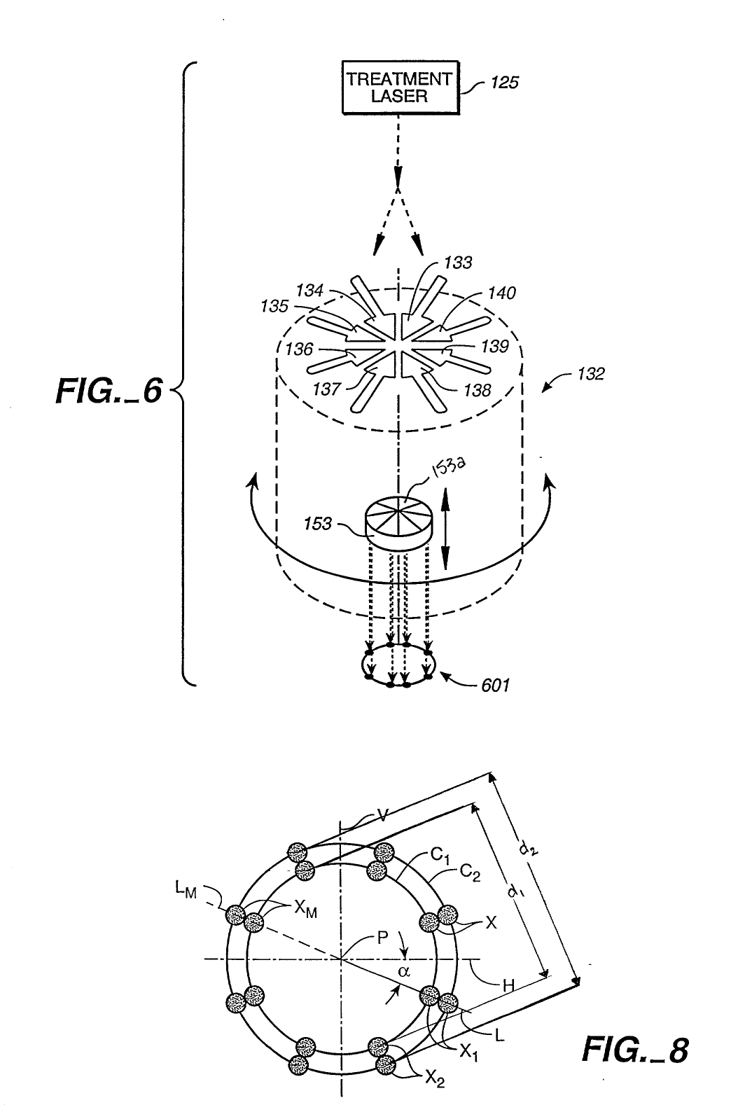

Before describing the optical system within the instrument 15 in some detail with respect to FIG. 6, a simplified schematic diagram of certain components of that optical system is provided in FIG. 3. Treatment and aiming laser radiation sources, along with radiation pattern adjusting optics, are indicated together as a block 93. Radiation from these sources is directed in a selected pattern as a beam 95 to a treatment dichroic mirror 97, where it is reflected along a path 99 through an objective lens 101 onto the eye 31 of the patient. The focusing spot projectors 81 and 83 are oriented for their beams to cross at the image surface of the objective lens 101. This surface is desired to be superimposed on the surface of the patient eye being treated. To focus the system in advance of performing a treatment, the instrument 15 is moved back and forth, toward and away from the patient, by the physician operating the handle 37 after the patient's head has been positioned in the heat rest 33 and immobilized by it. When the two beams of the focusing spot projectors 81 and 83 reflect from the eye as a single spot, the instrument is appropriately focused. If two spots are reflected, the physician knows that the system is not focused on the eye to be treated. The instrument 15 is then moved either away from or toward the patient, until a single spot indicating focus is obtained. The optical system also includes an LED 102 in the optical path 99. The patient is requested to fix his or her gaze on the LED 102 during preparation for and execution of the procedure.

The physician 17 views the patient's eye 31 along the optical path 99 through the mirror 97 and beam splitters 103 and 105. The beam splitter 103 directs a portion of the light returning along the path 99 into a video camera 107 that allows video recording of the procedure. A liquid crystal display (LCD) 109 is driven by the system computer and displays in one of the binoculars 27, but preferably not both, certain information of the treatment that is useful to the attending physician. This reduces the frequency of the instances when the attending physician needs to avert looking through the binoculars 27 during preparation for and during treatment. Information which can be presented in such a heads up display include the status of the system, magnitude of the headrest sensor 35 signal, whether or not the eye tracking system is locked onto the patient's eye, and the amount of treatment time remaining. This display is positioned in the physician's field of view so as not to interfere with a view of the patient's eye.

For photothermal keratoplasty, the wavelength of the treatment radiation is in the infra-red region of the electromagnetic radiation spectrum, use of a holmium doped yttrium-aluminum-garnet (Ho:YAG) crystal laser having a coherent radiation output with a wavelength of about 2.1 microns being preferred. Since this is not visible, an aiming light beam is superimposed on the treatment radiation beam. The physician then views in the visible region the form of the treatment radiation pattern. The aiming radiation can be in the red region and generated by a laser diode. The fixation LED is selected to emit in the green region, in this specific example. The illuminator need not be monochromatic and emit light in any region of the visible spectrum, but blue or green is preferred.

Referring to FIG. 4, the major visible elements of the patient's eye 31 are given for reference. Between upper and lower eyelids 111 and 113 are the sclera 115, iris 117 and pupil 119. Covering the iris and pupil is a transparent cornea whose outside curvature is changed by shrinkage of collagen within it according to the pattern of infra-red radiation directed against it. Referring to FIG. 5, an example is given of a region 121 in which treatment radiation is directed to correct for hyperopia and/or astigmatism. The region extends from an inner diameter d₂, which is about 5 mm (greater than that of the pupil, which is about 3 or 4 mm), to an outside diameter d₂ which is typically about 7 mm, but which can extend outwards to about 9 mm.

In the specific instrument example being described, the treatment radiation pattern at any one time has a maximum of eight radiation spots. The eight spots are symmetrically distributed around a circumference of a circle, separated from each other by 45 degrees. This circle has a center that is coincident with the center of the pupil 119 and a diameter dₛ that is adjustable between the diameters d₁ and d₂ of the exposure region 121. The rotatable position of the spot pattern is also adjustable to an angle 2 over a range of from −22.5 to +22.5 degrees from a radial reference 123. The diameter of each spot can also be made an adjustable parameter of the radiation pattern, although such an adjustment is not currently a feature of the system being described because it has been found to have little effect upon the results. Each of the eight spots is independently turned on and off in the instrument being described. A typical procedure includes multiple exposures to different patterns of spots. This allows the instrument to generate an almost unlimited number of different composite radiation patterns as the result of multiple exposures.

The primary optical elements of a sub-assembly 132 of the system being described is shown in more detail in FIG. 6. These optical elements may be utilized to form a circular pattern 601 of eight radiation spots, as a specific example. Radiation from a source 125, such as a laser source or a pulsed Ho:YAG treatment laser, is directed to the sub-assembly 132 that includes eight triangularly shaped shutters 133-140, one for each of the eight radiation spots that may be generated. These shutters are all shown to be closed, and include control motors (not shown) that individually operate the shutters between opened and closed positions. The sub-assembly 132 also contains a polyprism 153 that has eight substantially flat surfaces on one side that forms a pyramid, one of the eight treatment spots being formed from each of the flat surfaces. Radiation from the laser 125 is directed against the shutters and then, for those shutters that are opened, onto respective flat surfaces of the polyprism 153, through the polyprism and then through other optical elements not a part of the sub-assembly 132, and onto the patient's eye as a radiation pattern of up to eight spots. The eight spots are equally spaced around a circle. Of course, more or fewer than eight spots may be made available by use of a polyprism with a different number of flat surfaces, at least four such spots usually being desired.

For any particular treatment radiation exposure, the number of spots is controlled by opening the individual shutters 133-140 corresponding to the desired spot locations. The radius of the circle of spots 601 is controlled by moving the sub-assembly with respect to the laser 125. The angular positions of the spots around the circumference of the circle are controlled by rotating the sub-assembly 132. Additional details of the delivery optics and an associated control and operating system are given in the First Co-Pending Application referenced above.

The wavelength of radiation used for the treatment, its magnitude and the duration of the exposure, are selected to be adequately absorbed by the corneal or other exposed tissue to raise its temperature at the exposed spots to a level sufficient so that the tissue changes in the manner desired. When used to reshape the surface of the cornea or other tissue, the preferred technique is to control these parameters to cause tissue below the surface to change in a manner that reshapes the surface, without ablating the exposed tissue. The radiation wavelength is usually selected from the infra-red or near infra-red portions of the spectrum. However, the instruments and techniques described herein are also applicable to other processes that require exposure to radiation patterns with different parameters.

The above-described system is applied in this description to correct abnormal refractions of a patient's eye by delivering energy in a pattern of spots to the patient's cornea such that the curvature of the cornea is modified. The system can be used for a variety of refractive conditions, including astigmatism, and is particularly useful in the treatment of hyperopia wherein the curvature of the cornea is steepened to increase the refractive power of the cornea. Whatever the initial condition of the cornea, the treatment provider considers that condition, chooses an appropriate treatment plan, and treats the patient accordingly.

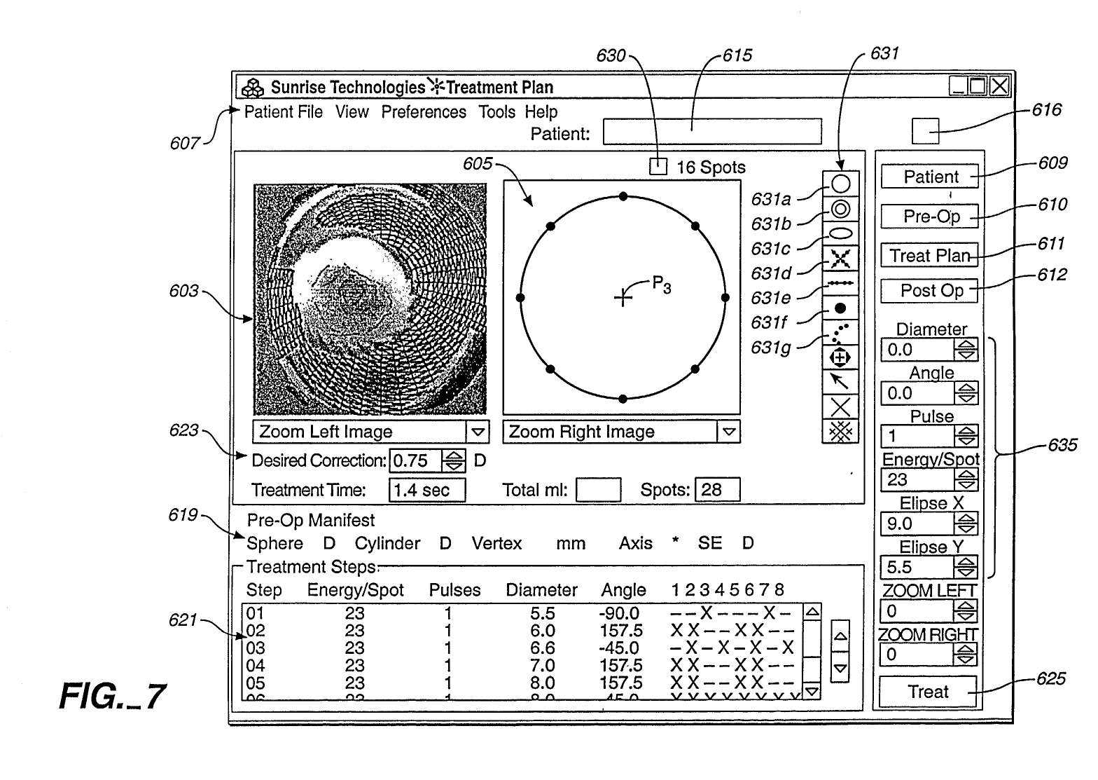

An example screen display used by the treatment provider to design and execute a treatment plan for a patient's eye is shown in FIG. 7 (“Treat Plan” screen). A central aspect of the display is the provision of two image boxes, a left image box 603 and right image box 605. In the example of FIG. 7, topographical representation of a patient's eye prior to treatment is displayed in the left box 603, and the pattern of radiation spots developed by the treatment provider (user) as part of a treatment plan for that eye is displayed in the right box 605. An important feature of the system being described is that the images of both of these boxes may be combined into one, thus enabling the user to designate the placement of radiation exposure spots at location across the eye where they will do the most good. Other items on the screen display of FIG. 7 are included in later descriptions of uses of the screen.

The planning of an appropriate vision-modification treatment and the carrying out that treatment is preferably interactive and automated, as further described below, using software that can be run on a desktop computer included as part of the ophthalmic treatment system. The primary interface is the touch screen display described above, which both provides information to the treatment provider (user) and accepts input commands from the user touching appropriate areas of the screen. A keyboard and a trackball, or a mouse, also provide a familiar method of data and command entry. The laser is preferably activated by using a foot switch, thus freeing the user's hands for interfacing with the touch screen, keyboard, trackball, and the patient. Additional details of the treatment system are given in the First Co-Pending Application referenced above.

The system software preferably utilizes screen displays and formats that are familiar or convenient to the user. By way of example, the software preferably uses “Windows”-type features, including layouts, menus, and control buttons, or icons, that are familiar to many, if not most, computer users. A control button on the screen may be activated physically, by touching or pressing the screen over the button, or by using the trackball to locate a screen pointer over the button and clicking an activation button on the trackball housing, as will be familiar to the user. The keyboard is used to enter patient information, treatment parameters, and the like.

Electrical power is preferably connected to the ophthalmic treatment system by activating a key switch, whereupon the system undergoes an automatic self-test. If the system fails the self-test, an alert, audio or otherwise, will be activated, and a system error will be indicated on the heads-up display. If the system passes the self-test, an initial screen or a main screen will appear on the touch screen display.

Typically, when the primary power is supplied to the system, the interactive tools of the system, such as the touch screen, trackball, and keyboard, are turned “on”. An initial “user log on” screen may be used for entry of the user's identification and password information to access the further screens and menus for patient-specific data entry, treatment planning, treatment, and the like. Once access is provided, a main screen that includes a button panel, a main menu, a toolbar, and a status bar appears. The user interacts with these features in a known manner using the interactive tools provided.

The status bar simply contains information concerning the status of the system software, such as what screen is open for use, as generally known. The toolbar contains typical “Windows”-type control buttons, or icons, for “New”, “Open”, “Save”, “Print”, and “Help”. Other icons may be included, such as the edit options of “Cut”, “Copy”, “Paste”, and the like.

The main menu typically offers “Patient”, “View”, “Preferences”, “Tools” and “Help” pull down menus, in an area 607 of the screen of FIG. 7. The “Patient” menu generally allows for selecting among the entry of patient-specific data, the opening of an existing patient data file, the saving of a patient data file, as well as exiting from the “Patient” menu. The “View” menu generally allows for toggling the status bar “on” and “off” and for making other choices affecting the form or content of the display. The “Preferences” menu typically allows for unit selection, such as metric units or other preferred units, for eye-chart preference selection, and for instrument- and treatment-preference selection. The “Tools” feature typically allows one to access service programs, or to connect to an Internet site, as described in the First Co-Pending Application referenced above, for enabling procedures. The “Help” menu generally allows the user to view information of the use of the software.

The button panel of many screens, such as the screen of FIG. 7, typically includes “Patient”, “Pre-Op”, “Treat Plan” and “Post-Op” input buttons 609-612, respectively. When touching or clicking upon the Patient or Pre-Op buttons 609 or 610, the treatment provider may create or view a patient file that includes the patient name, the identification of the left or right eye, and the pre-operative examination date. The screen of FIG. 7 displays the patient name and left or right eye in spaces 615 and 616. This patient file helps the user confirm that the pre-operative data is sufficiently current to serve as a basis for treatment planning, and that the treatment plan, treatment, and the post-operative information relate to the correct patient and the correct eye of that patient. Thus, preferably, the software is designed to require the creation of this patient file before the Treat Plan or Post-Op features are made available to the user.

As mentioned above, the patient file is created using the Patient and Pre-Op features. The Patient feature allows for the entry of patient-specific data, such as the patient name or other identifier, birth date, gender, contact data, and the like, or the viewing, editing, or saving of same. Preferably, the software is designed to require entry of either the patient name or identification, birth date and gender to create the patient file before the user may employ the Treat Plan or Post-Op features.

The Pre-Op feature allows the user to enter, view, edit, or save pre-operative examination data, such as the date of the examination and the name of the examiner; identification of the left or right eye; refractive information, for example, sphere (diopters) and cylinder (diopters), axis (degrees), and vertex (mm) for manifest, cycloplegic, and auto-cyclopegic; keratometry information, for example, steepness reading, flatness reading, and axis reading; other physical parameters of the eye, such as intraocular pressure (IOP, mmHg), pachymetry (Φm), and pupil diameter (mm); and standardized visual acuity charts, including uncorrected visual acuity (UCVA), best corrected visual acuity (BCVA), and uncorrected near visual acuity (UNVA), that can be selected in the Preferences pull down menu. A notes or commentary field is also provided for the examiner to enter text concerning the examination. Preferably, the software is designed to require entry or selection of the left or right eye of the patient to create the patient file.

Once the patient file is created, it may be used to assist the user in planning a vision-modification treatment for the selected eye of the patient. The user will verify certain information in the patient file, such as the patient name or identification and the eye to be treated, before accessing the Treat Plan feature for such planning.

The Treat Plan screen (FIG. 7 is an example of such a screen) may be accessed to view a treatment plan that has already been performed. In this case, the user will not be allowed to make changes in the Treat Plan screen. Alternately, the Treat Plan feature may be accessed for the planning of a new treatment. If the pre-operative examination data has already been entered for the patient eye to be treated, as described above, the Treat Plan screen will show the refractive data in a region 619 to assist the user in treatment planning. The manifest, cycloplegic, or auto-cycloplegic refractive data may be viewed according to a user's selection in the Preferences menu described above.

Based on the refractive data in the Pre-Op data file, a treatment plan (nomogram) is generated according to algorithms and default values provided in the system software. For example, the treatment plan including spots X (such as X₁, X₂, etc.) arranged in two concentric circles C₁ and C₂ of eight spots each may be generated, as shown in FIG. 8. The treatment plan also includes a desired correction, such as a spherical equivalent in diopters and an angle in degrees, that is generated from algorithms in the system software. The angle may be explained as follows. When a vertical axis V and horizontal axis H are drawn through the effective center point P of a proposed treatment pattern, the angle α corresponds to the angular separation between the horizontal axis H to the right of the center point P and a line L drawn from the center point through the first spot X₁ in the pattern beginning on the horizontal axis and moving in the clockwise direction therefrom. Preferably, the angle is from about zero (for example, if the first spot is on the horizontal axis) to about 45 degrees. For example, in the treatment pattern shown in FIG. 8, the angle α is about 22.5 degrees.

The treatment plan also includes the treatment parameters, such as the number of treatment steps, and for each step, the amount of energy (mJ) per spot, the number of energy pulses, the dimensions of the treatment pattern, those of the 8 possible spots which are to be used, and the like. The treatment plan is displayed in a region 621 of the screen of FIG. 7, where each line provides the parameters for a single exposure and the exposures are listed in the order that they are to be made. In the example just given with respect to FIG. 8 (not that shown in FIG. 7), there are two treatment exposures, one for the spots of each circle. For the first exposure, the parameters include 28 mJ per spot, 7 pulses, an effective pattern diameter d₁ of 6 mm, and all 8 of the spots being turned on. For the second exposure, the parameters include the same or different energy per spot, the same or different number of pulses, a different effective pattern diameter d₂, such as 7 mm, and all 8 spots being turned on. The software also optionally allows the user to simulate a treatment to get a simulated experience of how the treatment plan will be carried out.

In a specific example, in response to the user selecting a double circle pattern, the software generates spots in the two circles of FIG. 8 with respective diameters of 6 mm and 7 mm. Two exposures are necessary if 8 spots are in each circle, one exposure for each of the two circles. Four exposures are necessary if each circle has 16 spots. It is the energy level of the radiation spots in a predefined pattern that is varied to bring about the amount of tissue surface reshaping that is desired for a particular patient. When applied to a particular eye, the amount of desired correction ASE is determined by the physician and inputted in a field 623 (FIG. 7) to the system. The system software then calculates the amount of radiation energy that should be delivered to the cornea of that eye. For 8 spots in each circle, the total amount of energy, in mJ, for 8 spots is set equal to 237+(44.25)(ΔSE)−(0.60)(patient age). The patient age is among the data that are inputted to the system for each patient that is treated. For 16 spots in each circle, the total amount of energy, in mJ, for each 8 spots is set equal to 238+(15.95)(ΔSE)−(0.40)(patient age).

The treatment plan of a specific series of exposures is preferably alterable by the user after it is developed, especially when the plan has automatically resulted from execution of an algorithm in the system software from an entry of little more by the user than the desired correction in a display field 623 (FIG. 7). The user may interactively change this desired correction after a treatment plan has been established and view the new spot pattern replacing the old pattern in the right display box 605. When a changed correction is made by the user, the treatment plan is updated and re-calculated by the system software. In addition to the new pattern being displayed in the box 605, the details of the exposure steps displayed in the region 621 are updated. Alternately, the user may change certain specifics of the listed exposure steps by clicking on them and manually changing their values. This also results in the spot pattern displayed in the box 605 being updated if any changes affect the pattern.

Once the treatment plan is finalized, the user or treatment provider may prepare to treat the selected patient eye. Generally, the user ensures that the treatment plan information is correct. The user then presses a “Treat” control button 625 to gain access to a Treatment screen. Using the Treatment screen, the user may prepare the eye for treatment and commence treatment.

Typically, the user will precondition or dry the eye. Preferably, the eye is dried to reduce or eliminate a tear film that may otherwise compromise or interfere with the corneal-modification treatment. The upper and lower eye-lids may be held out of the corneal area, for example, using a speculum, to facilitate eye-drying. The eye may be dried using a flow of drying medium, such as warm (about 45° C.), dry air (≦about 15% relative humidity), for a period of from about 7 seconds to about one minute, and preferably, from about 15 seconds to about 30 seconds, as described in the Third Co-Pending Application. Preferably, the temperature and relative humidity are well-controlled, as may be accomplished using the conditioning system described in the above-mentioned application. This is the preferred conditioning method, as it minimizes eye-preparation time and provides substantially uniform eye-drying. Alternately, the eye may be dried naturally by the presence of ambient air in the vicinity of the eye over a period of time, such as three minutes. Natural drying is not preferred given the time involved and the lack of control over the ambient conditions, which may affect drying uniformity, and thus, the treatment outcome, and repeatability from one treatment to other treatments.

Whether the eye is dried naturally or using an eye-drying device, the user activates an eye-drying start control button to start a timer to time the eye-drying process. A default time for eye-drying may be selected in the Preferences pull down menu described above. Preferably, a drying-time countdown will appear in the Treatment screen, either showing the time remaining or the time that has expired in the eye-drying process.

Typically, the user activates an automatic calibration of the system, as described in the First Co-Pending Application referenced above, while the eye-drying is taking place. The automatic calibration process may be activated using an autocalibration control button on the Treatment screen. In the automatic calibration process, the system preforms a series of system calibrations and checks. Preferably, the Treatment screen includes a progress bar to show the progress of the automatic calibration and its success or lack of success. If the automatic calibration is unsuccessful, an explanatory message will be displayed and treatment will not be allowed. If the automatic calibration is successful, the user may proceed further with treatment preparation and treatment.

If a headrest is used with the system, as is preferred, the user will position the patient's head appropriately with respect to the headrest. A headrest assembly and its use are described in the Herekar et al. PCT Publication mentioned above. If a headrest is not used, the user will position the patient's head appropriately with respect to the ophthalmic treatment instrument. The user may then focus the microscope of the ophthalmic treatment instrument on the patient's eye, align or center the treatment pattern with respect to the eye, and look for an indication that the system is “Ready”. The Ready indications may appear on the heads-up display, as described in the First Co-Pending Application referenced above, and on the Treatment screen display in the form of an enabled Ready button. These Ready indications will appear or be enabled only when the automatic calibration has been successful and the eye-drying time has expired. If treatment is not performed within a certain time, such as about one minute, after the drying time expires, or within a certain time, such as about five minutes, after the automatic calibration is successfully completed, treatment of the eye will not be allowed. In such a case, the user will have to repeat the steps described above in preparation for treatment.

Once the system is in a Ready mode, the laser will be activated to deliver radiation continuously at the desired energy level while the safety shutter is closed to ensure that no energy reaches the patient. The user may set-up, check, or refine the head-positioning and the focusing and aligning of the ophthalmic treatment instrument with respect to the eye, as described above, prior to proceeding with treatment. By way of example, preferably the user will make sure the eye is within the visual field of the microscope, will confirm or adjust the centering of the treatment pattern with respect to the eye, and will confirm or adjust the focus so that two focus spots from a focusing laser (frequency doubled Nd:YAG or HeNe laser, for example) are brought together on the anterior surface of the cornea, as described in the First Co-Pending Application referenced above. Preferably, the user will also make sure that the head positioning is appropriate for treatment. For example, if a headrest assembly is used, the user will make sure the patient's head is pressed against the headrest, as described in the Herekar et al. PCT Publication referenced above.

The user may now begin treatment by activating a foot switch and maintaining it in an activated state to open the safety shutter and start the treatment, as described in the First Co-Pending Application referenced above. A “Treatment” indication will appear in a heads-up display viewable to the user when viewing the eye of the patient. Optionally, the user may activate an eye-tracker system to monitor eye movement during treatment, as described in the First Co-Pending Application referenced above, using an eye-tracker control button on the Treatment screen. The eye-tracker system monitors the eye during treatment, and, if the eye has moved more than a pre-defined amount (0.6 mm for example) since the delivery of the first treatment pulse, places the treatment procedure in a pause mode.

During treatment, the laser delivers radiation at the energy level specified for the first step in the treatment plan, for the specified number of pulses in the specified treatment pattern. This is shown to be the first line of the exposure data displayed in the region 621. The remaining exposures, one line at a time in order, are performed automatically by the system, one at a time, according to the treatment parameters specified for each exposure. The system automatically adjusts itself between exposures to provide the appropriate energy, number of pulses, angle α, circular diameter, selection of spots and the like, according to the data stored fore each of the exposures. This process is continued until all the specified exposures have been completed, at which time the eye has been treated according to the treatment plan.

Preferably, the Treatment screen shows the progress of the treatment by including the display 621 of the exposure steps with highlighting or some other marker indicating which of the lines of individual exposures is currently being, or just has been, made. The Treatment screen may also include a progress bar to show the progress of the treatment, for example, as a treatment-completion percentage. The Treatment screen may also include a status line for displaying prompts and messages to the user.

The user may put the treatment into a pause mode before all of the exposures have been made by deactivating the foot switch such that laser firing stops. The status of any pause may be displayed on the Treatment screen, such as the fact of the pause, the time elapsed in the pause mode, and the like. When the treatment is in a pause mode, the last exposure to have been made is highlighted in the display 621 and the progress bar, if any, shows the progress of the treatment until the pause mode took effect and will not show further progress until the treatment is resumed. Preferably, the Treatment screen will display a message providing brief instructions for resuming treatment. Treatment may be resumed by activating the foot switch within a certain time, such as ten seconds. If the treatment is not activated within the specified time limit, the system will go into a standby mode.

The treatment may also go into a pause mode if excessive eye movement is detected by an eye-tracker, if included in the system, as described above, or if the patient's head is moved out of an appropriate position with respect to the headrest assembly, as described in the Herekar et al. PCT Publication mentioned above. The user may decide to proceed with or without the eye-tracker, or with or without the head positioned appropriately with respect to the headrest assembly, respectively. To implement these decisions, the user either accepts the default mode or overrides the default mode using the interactive tools provided.

At the end of the treatment, a safety shutter is closed to prevent any further exposure to the laser radiation. Preferably, the treatment particulars are logged to the patient file, along with calibration data. The software system provides for the automatic archiving of patient and treatment data on a system hard mass storage disk drive. The system will provide a warning to the user if the archiving capacity drops below a certain level, such as 100 megabytes.

The user may create a post-operative examination record, using the PostOp feature. To create such a record, the user opens a patient file for the eye under examination and verifies that certain information in that patient file, such as patient name or identification and left or right eye, is correct. The user then presses or clicks upon the Post-Op button 612 to bring up the Post-Op screen. The Post-Op screen will be blank if no previous data has been entered, or will display the data from the last Post-Op record created for that particular patient eye. The Post-Op feature allows the user to enter posto-perative examination data using substantially the same data fields, such as examination date and examiner, refractive data, keratometry, visual acuity charts, IOP, pachymetry, and the like, as described above with respect to the Pre-Op feature. A pupil-diameter data field may be omitted, as the treatment should not cause any alteration (other than normal alterations depending on conditions such as light) in the diameter of the pupil of the patient's eye. A comparative data field may be added to display a change in refractive data since the last refractive examination. Notes may be entered in a similar manner to that used for the Pre-Op notes or commentary feature.

The Post-Op feature may also include a “Graph” option for showing the patient's progress based on pre-operation and/or post-operation data. For example, the graph may display refractive data, such as manifest refraction in diopters, chronologically from the date of the pre-operative examination to the date of at least one post-operative examination. Preferably, the graph is a Cartesian plot, with the refractive data (in ± diopters, for example) plotted with respect to the y-axis and the time data (in days, for example) plotted with respect to the x-axis.

As mentioned above, the Preferences menu may be used to set several system parameters and defaults according to the user's preference. The Preferences menu may include several files, one for each preference category. For example, the Preferences menu may include an “Eye Chart” file in which the user selects a visual acuity chart, such as a UCVA, BCVA, or UNVA chart. Once selected, these eye charts will be available by initializing the Pre-Op or Post-Op feature. The Preferences menu may include a “Refractions” file in which the user selects which refraction values from the Pre-Op feature, namely, manifest, cycloplegic, or auto-cycloplegic, will appear on the Treat Plan screen and be compared in the Post-Op comparative data field.

The Preferences menu may include an “Options” file in which the user selects various operational parameters, such as system defaults. For example, the user may select between “on” and “off” for the heads-up display, the eye-tracker, the aiming laser, the focusing laser, and the setting of a pause between treatment exposure steps. The user can override the eye-tracker default during operation, as previously described. In the Options file, the user may also set a default time for drying the eye before treatment. The Preferences menu may also include a “Date and Time” file, wherein the user selects a date format, such as month/day/year or day/month/year format, and a time format, such as a.m./p.m. or 24-hour format, and sets or adjusts the date and time, accordingly.

As described in the First Co-Pending Application referenced above, the system may be activated to enable an ophthalmic treatment procedure using the Internet or laser enablement cards. A Main screen displays an enablement value corresponding to the number of treatments that can be enabled. Each time a complete treatment is performed, the number of treatments remaining is reduced by one. Preferably, once the number of treatments remaining reaches ten, a reminder message is displayed. Once the number of treatments remaining reaches zero, no further treatments are allowed. As mentioned above, laser enablement cards may be used for system activation. Such a card is inserted into the system, as described in the First Co-Pending Application referenced above. Preferably, a laser enablement card can activate the system to enable only one procedure.

While FIG. 8 illustrates a possible treatment plan pattern, namely, a pattern of spots arranged in concentric circles, many other treatment plan patterns may be developed according to the present invention. A pattern illustrated in the display box 605 of FIG. 7, for instance, is a pattern of all eight spots arranged in a single circle. In addition, elliptical treatment plan patterns, such as those disclosed in the aforementioned Second Co-Pending Application, may be developed, particularly for the treatment of astigmatism at the same time that hyperopia is being corrected. These elliptical patterns may be achieved by arranging treatment spots in circular patterns of varying circular diameter (or radius) and effectively removing certain treatment spots from each of the circular patterns. The treatment plan is carried out by using the shutters 133-140 of the ophthalmic treatment system (FIG. 1) to prevent radiation delivery from certain spot locations in each circular pattern. Another possible pattern is a “bow-tie” pattern, as disclosed in the aforementioned Second Co-Pending Application, wherein two or more spots, such as three spots, are arranged together on opposite sides of a center point along two lines that pass through that center. This pattern is formed by the instrument of FIG. 1 with three successive exposures of spots in concentric circles of different diameters, all but four concentric spots being blocked by their respective apertures during each exposure.

While three pre-defined spot patterns have been described above, there are many patterns that may be developed to treat the eye of a patient. For example, the user may add or subtract individual spots or a group of spots from a pattern, such as the circular, elliptical and bow-tie patterns described above. Alternately, the user may place individual spots in the treatment region to form a more arbitrary treatment pattern. Also, the pre-defined spot patterns will be recognized as being symmetrical about a center of the circular patterns used to form them. Certain treatments, however, require non-symmetrical patterns to formed, where the pre-defined patterns are altered by adding or subtracting spots, as an example. Indeed, the eye represented by the topographical display in the region 603 (FIG. 7) has a non-symmetrical shape which requires a non-symmetrical pattern of radiation spots to reshape its corneal surface into a preferred spherical surface that is symmetrical about the center of the eye.

The user may enter certain pre-operative data for use in developing a treatment plan, as described above. For example, the user may import images of the patient eye, such as topographical, pachymetric, or other diagnostic images, for use in developing a treatment plan. The image is imported from the image source to the Treatment Plan screen described above. Several images may be so imported. The user may simply pull up an image for viewing it during the preparation of a treatment plan. Alternately, the user may place spots of a treatment plan pattern on one of these images, or on a grid system, using a graphical user interface. The spots may be placed anywhere in the treatment region, and thus, are not confined to placement on grid lines or the like.

Methods of developing a treatment pattern are fully described in the aforementioned Second Co-Pending Application. As disclosed therein, the user may develop a treatment plan pattern using an image or several images imported into the Treatment Plan screen or a grid. The grid may take a variety of forms, such as a bull's-eye-type grid having a center and concentric circles therearound, a graph-paper-type grid having parallel lines in vertical and horizontal directions, and the like. By way of example, a default grid may include a center point marked by cross-hairs and concentric circles therearound at the boundaries of the desired treatment region. This is what is shown in the image box 605 of the Treatment Plan screen of FIG. 7.

These techniques enable the user to enter or import diagnostic information, such as refractive data or imported topographic or pachymetric maps of the eye, and to develop a treatment plan pattern, such as a previously stored pattern or a newly generated pattern, based on the diagnostic information. For example, the user may develop a treatment plan pattern by placing spots over the imported image in the interactive image screen box 605 by using graphical, drawing and editing tools, as described in the Second Co-Pending Application. Once the user has developed the treatment plan pattern, the system is triggered to specify parameters of one or more exposures to spots in the available circular pattern that form the designed treatment plan pattern.

Use of a “Preference Settings” feature is also described in the Second Co-Pending Application. The system software is responsive to a user's input, for example, the user's development of a spot pattern. The software refines that input according to its algorithms, values, defaults, and the like, to generate preference settings. These preference settings may be viewed by the user in a Preference Settings screen, which is preferably interactive so that the user may change some of the preference settings, if so desired. The preference settings may include a range of diameter values for a circular pattern, from a minimum to a maximum value, such as from 5 mm to 9.5 mm. This preference setting corresponds to the preferred treatment region of the patient eye, namely, a region bounded by concentric circles, centered on the eye, the inner circle and outer circle having the minimum and the maximum diameter value, respectively. This preferred treatment zone excludes the central optical zone where treatment is not desired. A default treatment zone setting may be from a diameter of 4.5, 5.0 or 5.5 mm to about 8.5 mm. As this preference setting is based on a typical or average human eye, a user may adjust the setting, for example, if the patient has a non-typical eye. Adjustment of this and other preference settings may be accomplished by user interaction with edit features in the Preference Setting screen.

The preference settings may include information for specific patterns, such as ellipse, circle, and double-circle patterns, that are selectable from the button bar 631. For example, for an ellipse pattern, the effective diameter on the minor axis may be set at 5.5 mm, while that on the major axis may be set at 9.0 mm. Further by way of example, for a single-circle pattern, the circle diameter may be set at 8.0 mm, and for a double-circle pattern, the inner and outer circle diameters may be set at 6.0 mm and 7.0 mm, respectively. The preference settings may include energy and pulse information, such as a default energy per spot value and a default number of pulses. An energy per spot value of 23 mJ is an example of a possible default preference setting. An example of a default pulse number value is one. Preferably, the user may interact with the system to adjust the preference settings just described.

The preference settings may include defaults for a line pattern that is selected by activating the button 631e of the button bar 631. For example, the line pattern may have a default number of pairs of spots, such as two or three. A line pattern having two pairs of spots may be described in relation to FIG. 8, wherein the line pattern includes line L extending from the center P through two spots X₁ and dashed line L_{M} that mirrors line L and goes through two spots X_{M} that mirror the two spots X₁. The innermost spots X₁ and X_{M} relative to center point P form one pair of spots, while the remaining spots form the other pair of spots. The preference settings may include a default diameter value for the first pair of spots, such as 6.0 mm, and another for the second pair of spots, such as 7.0 mm. Preferably, the default number of pairs of spots and the default diameter value for each pair may be adjusted by the user.

The system software is designed to optimize the treatment plan pattern under development. The preference settings may include a default tolerance value for optimization of the pattern selected by the user. That is, the software will optimize the treatment plan pattern, such that the above-described aspects of the pattern, such as number of pulses, fall within a range of the preference setting ± the default tolerance value. Preferably, the default tolerance value may be adjusted by the user. If the values appearing in the Preference Settings screen are acceptable to the user, he or she may accept same interactively, such as by clicking on an “OK” feature or icon, or save same in a table file in a known manner.

Once the treatment pattern has been designed, the system software specifies parameters of one or more separate exposures, to be made in sequence on the cornea or other tissue being treated. The parameters of each exposure for a particular designed treatment are displayed in the exposure (optimization) table 621 (FIG. 7), as described above. Since the instrument of FIG. 1 only generates multiple spots in a circular pattern, two or more exposures to selected spots with different geometric parameters are most commonly made in order to expose the cornea or other tissue surface to the designed treatment plan pattern. The numbers and specifics of the exposures that form each of several of common patterns are described above. The system software references a table when an elliptical pattern is specified by the user, for example, to obtain default parameters of each of multiple circular spot pattern exposures that forms the specified ellipse.

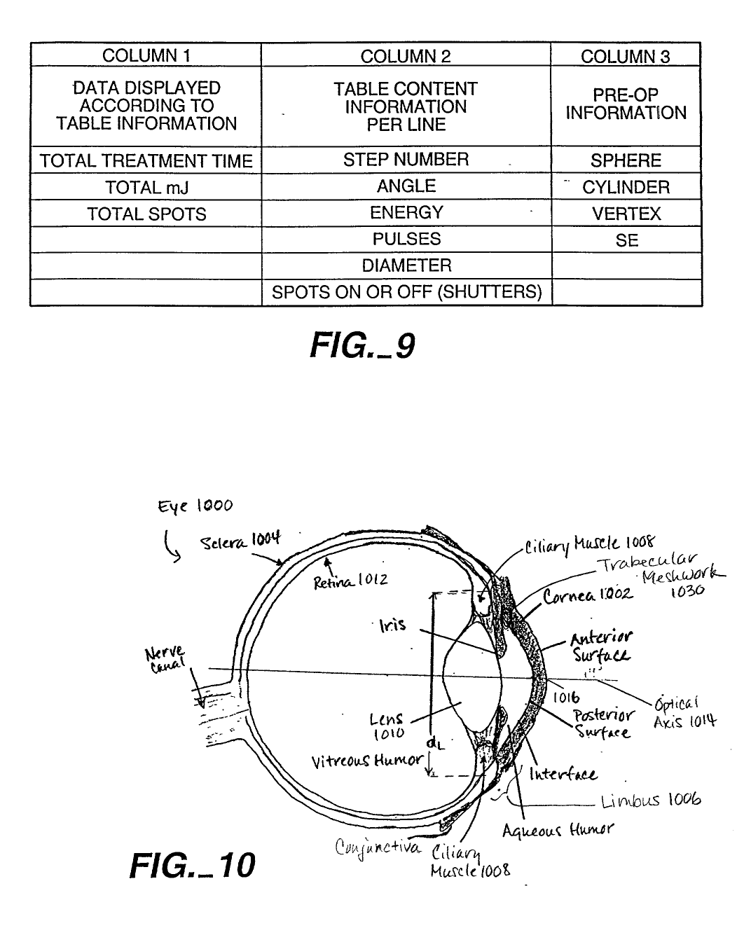

A method for changing parameters of a selected treatment plan spot or pattern are disclosed in the Second Co-Pending Application. If a change is made, the values for the selected spot or pattern and the image screen are updated by the system. Further, an optimization (exposure step) table is updated by the system, as also described in the Second Co-Pending Application. Preferably, the optimization table appears in the Treatment Plan screen area 621, where the user is interactively generating or has so generated a treatment plan pattern. During this process, the system responds with a table update, or optimization table update. Preferably, the optimization table is a table-form arrangement of the display fields shown in Column 2 of the “System Display Fields” table of FIG. 9. While the fields may be displayed in any order or table format, it is preferable to arrange the fields in columns, starting with a column for the step (exposure) numbers and rows therebeneath displaying the step number, beginning with the first step of the pattern. The remaining information will then be displayed in adjacent columns, with the information displayed in rows therebeneath, wherein the information in a particular row corresponds to the step number displayed at the beginning of that row. By way of example, the table may be of the form illustrated in region 621 of the Treatment Plan screen (FIG. 7) with columns for displaying the energy, such as the energy per spot, the pulses, such as the number of pulses, the effective pattern diameter at the patient eye, the angle α, and the spots that are “on” and “off” in the pattern. As to the latter, the column may be split into 8 or 16 sub-columns, with crosses (“x”s) or dashes (“-”s) under “on” spots or “off” spots, respectively. A “Desired Correction” value will also be displayed in the Treatment Plan screen in display field 623, typically, apart from the optimization table.

Other data may be displayed in the Treatment Plan screen, such as that shown in Columns 1 and 3 of the table of FIG. 9. For example, the total treatment time, total energy, and total number of spots for the treatment plan pattern may be displayed, in any desirable format. The system automatically generates this data from the data in the optimization table. Further by way of example, pre-operative data described above, such as the sphere, cylinder, vertex, and spherical equivalent, whether manifest, cycloplegic, or auto-cycloplegic, may be displayed in the Treatment Plan screen in any desirable format. The user may interact with the optimization table and the Desired Correction feature, as disclosed in the Second Co-Pending Application. That is, if the user wishes to change the desired correction value displayed in the Treatment Plan Screen, he or she may change it using an interactive tool, such as an increment/decrement feature. The system will respond by automatically updating the energy value of that treatment exposure, according to the system software, and displaying the updated energy value in the optimization table. The various menu options available to the user to accomplish many of the foregoing treatment planning steps and methods are described further in the Second Co-Pending Application.

An example of preferred computer software to implement the foregoing is provided in source code in the microfiche appendix that is part of the disclosure herein. This source code is subject to copyright protection by Sunrise Technologies International, Inc., assignee of the present application. The copyright owner has no objection to the facsimile reproduction by anyone of that appendix, as it appears in the Patent and Trademark Office patent file or records, but otherwise reserves all copyright rights whatsoever.

The apparatus and methods described above may be applied to the treatment of presbyopia, as will now be described in relation to FIG. 10. FIG. 10 shows an eye 1000, an optical axis 1014 drawn through the center of the cornea 1016 and the overall eye, and various components of the eye, including the cornea 1002, the sclera 1004, the limbus 1006, the ciliary muscle 1008, the crystalline lens 1010, and the retina 1012. The limbus is located near the point of attachment of the ciliary muscle 1008 to the sclera 1004. In the human eye, the limbal diameter d_{L} across the center of the eye is typically about 11 to about 12 mm.

It has been shown that in animal eyes, no refractive change takes place when LTK is performed within a region bounded by a radius from the center of the eye of greater than about 4.1 mm, or a diameter across the center of the eye of greater than about 8.2 mm. It is believed that the spherical shell of the eye within this region is functionally rigid with respect to LTK treatment performed within a region bounded by a radius of greater than about 4.25 mm, or a diameter of greater than about 8.5 mm.

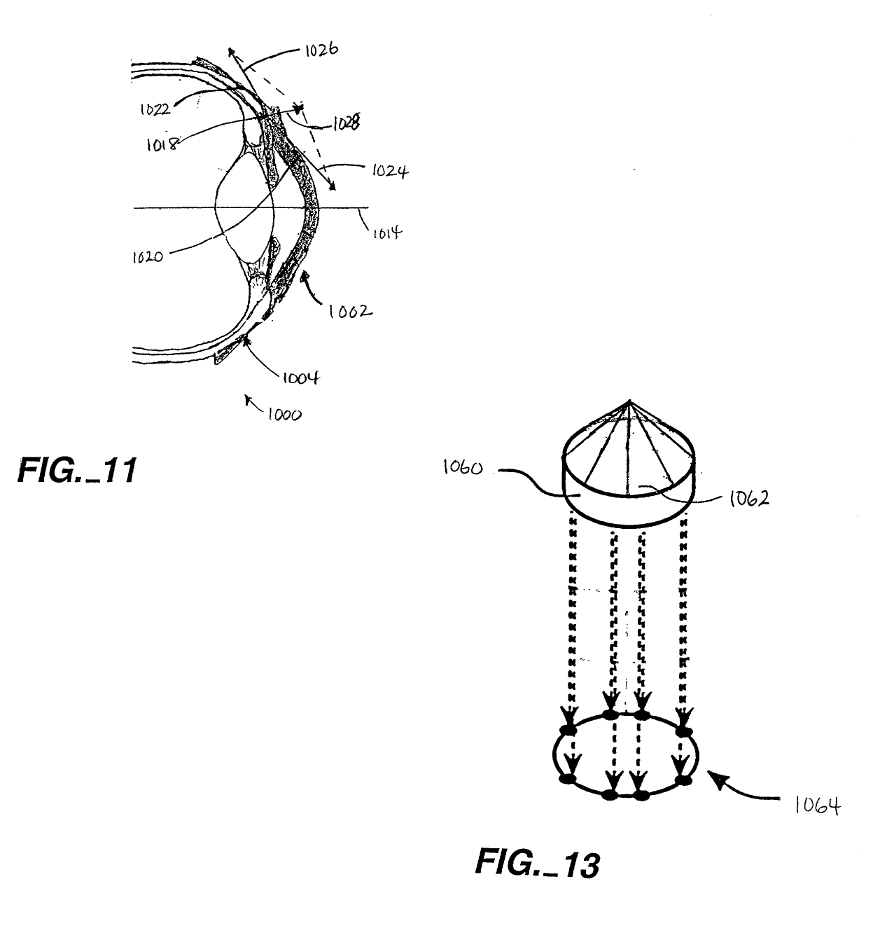

According to the present invention, a collagen tissue shrinkage procedure, as previously described, is performed near the point of attachment of the ciliary muscle 1008 to the sclera 1004. It is believed that a tensile force is generally associated with lo collagen tissue shrinkage. Further, it is believed that a tensile force resulting from a collagen tissue shrinkage procedure performed near the point of attachment of the ciliary muscle 1008 to the sclera 1004 will be directed outward from the eye and will induce a motion of corneal and/or scleral tissue in such an outward direction, away from the pre-treatment position of such tissue, as schematically illustrated in the vector diagram of FIG. 11.

As shown in the schematic illustration of FIG. 11, ocular tissue may be exposed during a collagen shrinkage procedure at locations near the point of attachment 1018 of the ciliary muscle 1008 to the limbus 1006, such as at corneal location 1020, at scleral location 1022, or both. When location 1020 or location 1022 is selected, a force, or tensile force, along force vector 1024 or 1026, respectively, results, causing movement of the limbal tissue, and the corneal or scleral tissue between the limbus and the exposure location, in the direction of the vector. When both locations 1020 and 1022 are selected, a net force vector 1028 results, causing movement of the limbal tissue, and the corneal or scleral tissue between the limbus and the exposure locations, in the direction of that vector. The ultimate result of any of these movements is a displacement of limbal tissue, as well as corneal and/or scleral tissue, away from the crystalline lens 1010. The therapeutic result is an increase in the accommodative ability of the ocular tissue, and particularly the lens, for near vision.

According to an aspect of the present invention, the collagen tissue is exposed at the selected location to heat or radiation sufficient to raise the collagen temperature to a shrinkage temperature of from about 55° C. to about 100° C., preferably, from about 55° C. to about 70° C., in the manner disclosed herein above and in the First-Fifth Co-Pending Applications mentioned above. Any suitable heat or radiation source may be used, such as a laser, a source of incoherent radiation, a source of radiofrequency radiation, a source of microwave radiation, a source of ultrasonic radiation, and a source of thermal radiation, such as a tissue-contacting source of thermal radiation, or an electrical source of thermal radiation. The source of heat may be pulsed or intermittent, or continuous. Preferably, the source is a laser, such as a pulsed laser emitting radiation of a wavelength of from about 1.4 to about 2.55 microns, such as from about 1.8 to 2.55 microns, or a pulsed Ho:YAG laser. The treatment energy may any at any safe and efficacious energy level up to the capacity of the system, such as up to about 300 mJ per eight spots. Generally, a safe level is that which avoids necrosis of the corneal or scleral tissue, where the precise energy density will naturally depend on the spot diameter. Upon heat exposure, the collagen tissue reaching the shrinkage temperature will shrink. Collagen fibrils have been reported to shrink to as little as ⅓ of their pre-treatment length when heated to shrinkage temperature.

In localized regions of corneal collagen tissue, the corneal structure prevents such large changes. Thus, for best results, this factor should be taken into account in the development of the treatment plan for the shrinkage of corneal collage tissue. By way of example, consider collagen shrinkage treatment of a peripheral region of the cornea, such as a ring centered at the center point 1016 of the cornea along axis 1014 and having a diameter of about 10 mm. The circumference of such a region is typically about 31.4 mm. Consider further treating about half of this region, or about 15.7 mm of tissue along the ring, using an annular pattern of spots corresponding to tissue along the ring. If the diameter of each spot in the pattern were about 0.6 mm at the corneal surface, where the diameter may be selected from about 0.4 mm to about 1.0 mm, a pattern of about 26 spots (15.7/0.6) might be used. In such a case, shrinkage of the collagen fibrils by ⅓ (half of the amount reported in in vitro tests) would correspond to shrinkage of the diameter of the annular treatment region by about 0.2 mm. If only half of the circumferential region were treated (i.e., using half the number of exposure spots in the annular treatment region), the net shrinkage might be about 0.1 mm. Further, if the tissue in the annular treatment region were to move a distance that is about half of that net shrinkage, the tissue movement would be on the order of about 50 microns, a movement expected to be of clinical significance.

It will be understood that the foregoing considerations simply provide an example of a possible treatment plan, as there are numerous possible treatments which effectively account for variations in the treatment parameters. By way of example, treatment parameters subject to variation include the selection of a single treatment region or a number of treatment regions, the size of a treatment region, the shape of a treatment region, the pattern used to treat a treatment region, such as the number or size of spots in the pattern, the intensity of irradiation, the duration of irradiation, the selection of a single pulse of radiation or a number of pulses of radiation from a pulsed source, the selection of a continuous source of radiation, as well as various other treatment parameters.

Very similar considerations may be applied in the development of a treatment plan for collagen shrinkage in the sclera. By way of example, consider collagen shrinkage treatment of a region of the sclera, such as a ring centered at the center point 1016 of the cornea along axis 1014 and having a diameter of from about 14 to about 22 mm, such as from about 17 to about 22 mm. The circumference of such a region will be considerably larger than that of the corneal region considered above, such that to treat about half of the region with a pattern of 0.6 mm-diameter spots, many more spots will be needed. The exact number of spots will depend on the selected diameter of the region, the diameter of the spots, and the like.

An effective plan for treating the sclera may be selected from any of numerous possible treatments which effectively account for variations in treatment parameters, such as those previously described. When selecting such a treatment plan, it should be noted that it is important to leave the ocular tissue in a vicinity of the trabecular meshwork 1030 substantially intact. Thus, thermal treatment of such ocular tissue is preferably minimal or insignificant, or more preferably, substantially avoided. It has been noted that scleral expansion enlarges the pore size in the trabecular meshwork, thus lowering intraocular pressure (IOP). LiVecchi, Presbyopia: The Final Frontier, ASCRS Symposium p.84, (May 2000). This may be a particular advantage of the scleral treatment procedure of the present invention when the subject undergoing treatment evidences elevated IOP.

Considerations like those described above may also come into play in the development of a treatment plan including both corneal and scleral treatment. Based on the vector diagram of FIG. 11, it is anticipated that the result of such a hybrid treatment may be at least twice as great as that for either the corneal or scleral treatment alone. When anticipating the results of a hybrid treatment, however, it should be noted that the behavior of the tissue undergoing treatment may not necessarily be linear, uniform, or elastic. As the motion of the tissue, or the strain experienced by the tissue, may not be linear, the ultimate motion of the tissue may not be proportional to the hybrid treatment, and in particular, may depart from the result expected from that treatment. For example, the ultimate motion of the tissue may vary from that expected from the hybrid treatment in that the limbus may be moved closer to, or farther from, the periphery of the lens than expected.

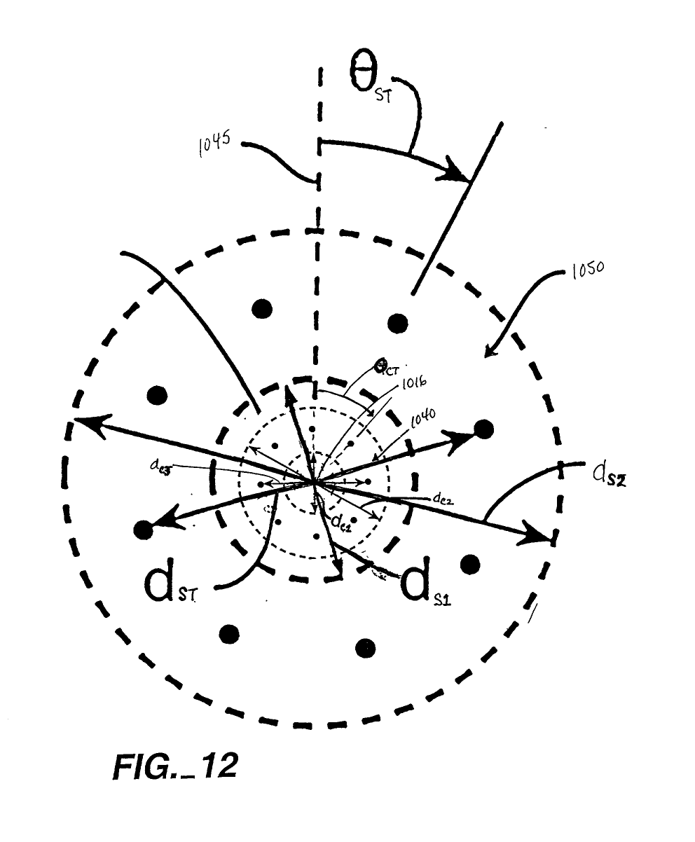

The corneal and scleral treatments just described are now further described in relation to FIG. 12. FIG. 12 shows a corneal region 1040 which may be selected for the corneal treatment described above for the treatment of presbyopia, or the treatment of near vision accommodation deficiencies. This corneal region 1040 extends from an inner diameter d_{C}, across the center point 1016 of the cornea of about 8 to about 9 (preferred) mm, or a radial distance from the center point of about 4 to about 4.5 (preferred) mm, to an outer diameter d_{C2} across the center point of about 11 (preferred) to about 12 mm, or a radial distance from the center point of about 5.5 (preferred) to about 6 mm. According to the corneal treatment, heat or radiation, whether in the form of a single spot or other shape, or an intermittent or continuous pattern of spots or other shapes, is directed to an anterior surface of corneal tissue within the corneal region (for example, at a treatment diameter d_{CT}) to bring about the desired collagen shrinkage, much in the manner described in relation to FIG. 5. While a pattern of eight spots is shown in FIG. 12, this pattern is merely an example of a possible treatment pattern, such that a different pattern or a different number of spots, such as from about 8 to about 32 spots, or such as 24 or more spots, may be selected for the corneal treatment for improving near vision accommodation. The pattern may be adjusted in terms of angular rotation, as represented by a rotation angle 2_{CT} of from about −22.5 to about +22.5 degrees from a radial reference 1045, much in the manner described in relation to the rotation angle 2 shown in FIG. 5.

FIG. 12 also shows a scleral region 1050 which may be selected for the scleral treatment described above for the treatment of presbyopia, or the treatment of near vision accommodation deficiencies. This scleral region 1050 extends from an inner diameter d_{S1} across the center point 1016 of the cornea of about 12 mm, or a radial distance from the center point of about 6 mm, to an outer diameter d_{S2} across the center point of about 20 mm or about 22 mm, or a radial distance from the center point of about 10 mm or about 11 mm. According to the scleral treatment, heat or radiation, whether in the form of a single spot or other shape, or an intermittent or continuous pattern of spots or other shapes, is directed to an anterior surface of scleral tissue within the scleral region (for example, at a treatment diameter d_{ST}) to bring about the desired shrinkage of collagen tissue within the sclera, much in the manner described in relation to FIG. 5. While a pattern of eight spots is shown in FIG. 12, this pattern is merely an example of a possible treatment pattern, such that a different pattern or a different number of spots, such as from about 8 to about 64 spots, or such as about 40 or more spots, may be selected for the scleral treatment for improving near vision accommodation. The pattern may be adjusted in terms of angular rotation, as represented by a rotation angle d_{S2} of from about −22.5 to about +22.5 degrees from a radial reference 1045, much in the manner described in relation to the rotation angle 2 shown in FIG. 5.

Either one or both of the corneal and scleral treatments just described may be used to treat ocular tissue for correcting or improving upon a near vision accommodation deficiency. In the corneal treatment, heating of the collagen tissue to a shrinkage temperature of from about 55° C. or about 60° C. to about 70° C. or about 80° C., or from about 68° C. to about 80° C., is preferred, as heating the tissue to temperatures beyond 80° C. may cause a corneal haze, opacity, or cloudiness that may affect vision. In the scleral treatment, these temperature ranges can be increased to have an upper temperature of about 100° C., as haze is not much of a concern, if any, in the scleral region. Successful treatment of a near vision accommodation deficiency may be reflected in an improved uncorrected visual acuity (UNVA) reading, or by the patient's ability to read without unnatural aids such as glasses, contact lenses, and the like. It is believed that successful treatment would provide a refractive accommodation in a range of up to and including about 2 diopters, with a very successful treatment providing a refractive accommodation further up to and including about 3 diopters.

A person with poor accommodative ability might have 20/20 vision for distant objects at about 20 feet (about 6 meters) or more, but poor vision for near objects at about 16 inches (about 40 centimeters). Near vision is measured in a number of ways, Snellen Distance Equivalent and Jaeger systems being quite commonly used. A person with poor near vision might have a Snellen near value of 20/63 or a Jaeger near value of J9. Minimum improvement would be one “line” or increment to Snellen 10/50, or Jaeger J7. A substantial improvement would be about three “lines” to Snellen 20/32, or Jaeger J3. A very successful treatment would bring this patient to Snellen 20/20, or Jauger J1, for near vision, an improvement of five “lines”, which corresponds to about 2 to about 3 diopters of refractive correction.

In either the corneal or the scleral treatment, the source of heat or radiation, whether continuous or pulsed, produces at least one region of heating or radiation which corresponds to a treatment region, whether a corneal region 1040, a scleral region 1050, or both, where collagen shrinkage is desired. By way of example, a substantially continuous region of heating or radiation, such as a substantially continuous annular region, may be used to form a selected pattern. According to another example, overlapping regions of heating or radiation may be used to form a particular treatment pattern. The system previously described herein is designed or adapted to provide patterns based on eight or multiples of eight spots, arranged in substantially annular manner, although a suitable system according to the present invention may be designed or adapted to provide other patterns as described herein and in the First-Fifth Co-Pending Applications mentioned above.

The corneal treatment, and of course the scleral treatment, are directed at regions that are sufficiently remote from the central optical zone, such that significant refractive change in the cornea is not anticipated. Thus, these treatments may be performed on almost any patient, regardless of the refractive condition of his or her vision. When the treatment of the corneal or scleral regions is a LTK treatment, the treatment is expected to be quite safe based on safety evaluations of LTK treatments of corneal tissue for hyperopia. Preferably, a local anesthetic will be used prior to LTK treatment of corneal tissue. It is unclear whether or not such anesthetic preparation will be particularly desirable prior to LTK treatment of scleral tissue, although such preparation may be used.

The corneal, scleral, or hybrid treatments described above are expected to produce desirable improvements in near vision accommodation. Even a modest improvement in accommodation is highly desirable for patients whose accommodative ability may have dropped significantly, such as to a level of nearly zero as is typical in patients of about 60 years of age or more. By way of example, such a modest improvement would provide a refractive accommodation in a range of up to and including about 1 diopter of refractive accommodation (or about 3 lines on the Snellen scale). Further by way of example, a modest improvement may be marked by a patient's ability to read with more moderate aids, such as moving from trifocal to bifocal aids, and the like. In cases in which the effect of the treatment lasts for a shorter period than desired, it may be desirable to retreat the tissue.

The corneal, scleral, and hybrid treatments described above may be carried out using various treatment systems. Preferably, a system quite similar to that of FIG. 1, as described herein and in the First and Second Co-Pending Applications mentioned above, is used to provide infrared laser energy in a pattern of spots, such as eight spots, simultaneously, to the target tissue. That is, the system of FIG. 1, and particularly the optical system thereof, is modified somewhat to allow treatment at diameters greater than those described in relation to FIGS. 5 and 8. This modification is now described in relation to the optical system shown in FIG. 6.

FIG. 6 shows an optical system which includes a polyprism 153 having a number of flat surfaces 153a on one side that are angled to form a pyramid. In the modification of this optical system for the treatments just described, the polyprism is modified such that its flat surfaces are more steeply angled to form the pyramid. FIG. 13 shows such a modified polyprism 1060, having flat surfaces 1062 on one side that are steeply angled to form a pyramid. When this modified polyprism 1060 is substituted for that shown in FIG. 6, the radiation leaving the polyprism will form a pattern 1064 having a greater diameter than the pattern 601 of FIG. 6.

While polyprisms of various steepness (or flatness) may be used, it is desirable to use a single polyprism that can accommodate all of the desired treatments, namely, the corneal treatment, the scleral treatment, and the hybrid treatment described above. Such a polyprism might be a polyprism of adjustable steepness, if practical, or a polyprism having a set steepness that is sufficient for all of these treatments. The diameter of pattern 601 (FIG. 6) is about half of the maximum diameter contemplated for the scleral or corneal-scleral hybrid treatments described above, such that the steepness of the pyramid polyprism 1060 is preferably on the order of about twice that of the pyramid polyprism 153 of FIG. 6. Such a polyprism 1060 is suitable for a wide range of treatments, from hyperopic treatments at a minimum diameter of about 5 mm to scleral or hybrid treatments at a maximum diameter of about 22 mm.

Treatments at the larger diameters may not be in focus if the normal focusing system of the ophthalmic treatment system of FIG. 1, as described in the First Co-Pending Application, is used. This may not pose a problem for near vision accommodation treatments, as precise refractive changes are not sought in these in these treatments. Nonetheless, it may be desirable to move the focus of the radiation beams from the anterior surface of the targeted tissue to a deeper or more posterior point, to provide sufficient fluence at the targeted tissue. This might be accomplished by any of a variety of suitable methods, such as by modifying the focusing system described in the First Co-Pending Application, so that reference points for the green focus beams are provided on the eyepiece reticle, so that a direction-changing device is placed in the path of the green focus beams, or the like.

While the invention has been discussed with particular reference to treatments in the corneal and/or scleral tissues of the eye, the invention has application to a variety of ophthalmic tissues and non-ophthalmic bodily tissues. As to the former, for example, it is contemplated that the eye can be treated according to the invention by applying heat or radiation to the conjunctival tissue of the eye. It is believed that the conjunctiva is thin enough such that this heating of the conjunctiva will penetrate tissue underlying the conjunctiva, such as the scleral tissue, to cause shrinkage of the collagen 40 of the conjunctiva and/or the underlying tissue. Other ophthalmic applications include treatments associated with a corneal transplantation or a pre- or post-surgical treatment of ophthalmic tissue, including touch-up or re-treatment. As to the latter, the invention may be used to treat non-ophthalmic tissue by simply positioning the treatment system relative to the target tissue and proceeding with the treatment of that tissue. The invention thus has many useful applications including a great variety of treatments for correcting an undesirable condition of selected tissue by modifying a shape, structure, or appearance of the tissue being treated. By way of example, the invention may be used to treat a tissue wound, a surgical site, tissue having an undesirable condition, cosmetic, medical or otherwise. In a particular example, it is contemplated that cosmetic conditions of the skin, such as undesirable wrinkles, can be treated according to the invention by applying heat or radiation to the epidermis to cause shrinkage of the collagen of the epidermis and/or tissues therebeneath.

Various aspects and features of the present invention have been explained or described in relation to beliefs or theories, although it will be understood that the invention is not bound to any particular belief or theory. Further, although the various aspects of the present invention have been described with respect to the preferred embodiments thereof, it will be understood that the invention is entitled to protection within the full scope of the appended claims.

It is claimed:

1. A method of treating ocular tissue of an eye defining a central optical axis therethrough, comprising:
providing a pattern of heating according to which the ocular tissue is to be exposed, the pattern including at least one region of heating that corresponds to an ocular region within a radial distance of from about 4 mm to about 11 mm from the optical axis, and the heating sufficient to shrink collagen tissue within the ocular tissue; and
exposing the ocular tissue to the heating according to the corneal pattern to shrink collagen tissue within the ocular region thereby improving accommodation for near vision.

2. The method of claim 1, wherein the ocular tissue is corneal tissue and the pattern includes at least one region of heating that corresponds to a corneal region within a radial distance of from about 4 mm to about 6 mm from the optical axis.

3. The method of claim 1, wherein the ocular tissue is scleral tissue and the pattern includes at least one region of heating that corresponds to a scleral region within a radial distance of from about 6 mm to about 11 mm from the optical axis.

4. The method of claim 1, wherein the exposing of ocular tissue includes exposing the ocular tissue in a vicinity of a region in which ciliary muscle attaches to the ocular tissue.

5. The method of claim 1, wherein the exposing of ocular tissue causes the sclera to move in a direction away from an ocular lens of the eye.

6. The method of claim 1, wherein the exposing of ocular tissue creates a force in a direction outward from a limbus of the eye.

7. The method of claim 1, wherein the exposing of ocular tissue raises a temperature of collagen tissue within the exposed region to from about 55° C. to about 100° C.

8. The method of claim 1, wherein the exposing of ocular tissue raises a temperature of collagen tissue within the exposed region to from about 55° C. to about 70° C.

9. The method of claim 1, wherein the exposing of ocular tissue includes exposing the ocular tissue to heating from a source selected from a group consisting of a source of incoherent radiation, a source of radiofrequency radiation, a source of microwave radiation, a source of ultrasonic radiation, a tissue-contact source of thermal radiation, an electrical source of thermal radiation, a source of infrared radiation, a laser, and any combination thereof.

10. The method of claim 9, wherein the source is selected from a group consisting of a pulsed source and a continuous source.

11. The method of claim 1, wherein the exposing of ocular tissue improves a condition of presbyopia.

12. The method of claim 1, wherein a parameter of the exposing of ocular tissue is selected from a group consisting of a singular treatment region or a number of treatment regions, a size of a treatment region, a shape of a treatment region, a wavelength of radiation, an intensity of radiation, an intensity of heating, a singular pulse of radiation or a number of pulses of radiation from a pulsed source, a selection of a continuous source of heating, a period of exposure, and any combination thereof.

13. The method of claim 1, wherein the pattern is a pattern of radiation spots.

14. The method of claim 13, wherein the radiation spots have an effective diameter of about 0.4 mm to about 1.0 mm at a surface of the ocular tissue.

15. The method of claim 13, wherein a number of radiation spots is selected from a group consisting of eight or multiples of eight.

16. The method of claim 2, wherein the corneal pattern is a pattern of spots and a number of radiation spots is from about 8 to about 32.

17. The method of claim 3, wherein the scleral pattern is a pattern of spots and a number of radiation spots is from about 8 to about 64.

18. The method of claim 1, wherein an intraocular pressure of the eye is reduced.

19. The method of claim 1, wherein radiation of ocular tissue in a vicinity of trabecular meshwork of the eye is substantially avoided.

20. The method of claim 1, wherein the pattern is a substantially annular pattern.

21. The method of claim 20, wherein a source of the annular pattern is selected from a group consisting of a continuous source of the at least one region of heating and a pulsed source of the at least one region of heating.

22. The method of claim 20, wherein the substantially annular pattern is comprised of at least one region of radiation forming a substantially continuous annulus.

23. The method of claim 20, wherein the substantially annular pattern is comprised of overlapping regions of radiation.

24. The method of claim 1, wherein accommodation for near vision is improved by up to and including about 3 diopters.

25. The method of claim 1, wherein accommodation for near vision is improved by from one to five lines Snellen or Jaeger, or an amount corresponding thereto.
